(19) United States
(12) Patent Application Publication
(10) Pub. No.: US 2020/0138267 A1
(43) Pub. Date: May 7, 2020

(54) ENDOSCOPE SYSTEM

(71) Applicant: OLYMPUS CORPORATION, Tokyo (JP)

(72) Inventors: Sadaaki TOMURA, Tokyo (JP); Soichiro IMOTO, Kawasaki-shi (JP); Toshiyuki FUJII, Tokyo (JP)

(73) Assignee: OLYMPUS CORPORATION, Tokyo (JP)

(21) Appl. No.: 16/733,940

(22) Filed: Jan. 3, 2020

Related U.S. Application Data
(63) Continuation of application No. PCT/JP2018/030029, filed on Aug. 10, 2018.

(30) Foreign Application Priority Data
Aug. 25, 2017 (JP) 2017-162784

(51) Int. Cl. A61B 1/00; A61B 1/07; A61B 1/12; G01K 13/00

(52) U.S. Cl. CPC A61B 1/00009; A61B 1/00096; G01K 13/002; A61B 1/07; A61B 1/128; A61B 1/00027

(57) ABSTRACT

An endoscope system includes: an endoscope including an insertion portion whose front end is inserted inside a subject; a heater disposed at the front end and configured to heat a predetermined member disposed at the front end; a plurality of thermometers disposed near the predetermined member at the front end and configured to detect temperatures at the front end; a power source configured to supply electrical power to the heater; determination circuitry configured to determine whether or not highest temperature, from among the temperatures detected by the plurality of thermometers, is equal to or higher than a first threshold value; and a power controller configured to control, based on determination result of the determination circuitry, the electrical power supplied by the power source to the heater.

This application is a continuation of PCT International Application No. PCT/JP2018/030029 filed on Aug. 10, 2018, which designates the United States, incorporated herein by reference, and which claims the benefit of priority from Japanese Patent Application No. 2017-162784, filed on Aug. 25, 2017, incorporated herein by reference.

### BACKGROUND

The present disclosure relates to an endoscope system that is inserted inside a subject for taking images of the subject and generating image signals.

In the related art, endoscopes are known that are inserted inside a subject for observing the regions to be tested, and the endoscopes are widely used in the medical field. An endoscope is used inside a body in which the humidity is high and the temperature is higher than the room temperature. Hence, when the front end of the insertion portion of an endoscope is inserted inside a body, it results in the clouding of the optical members such as a lens cover and an objective lens installed at the front end; and there are times when it is not possible to obtain clear images. For that reason, in a known endoscope, a technology is known in which a heating unit such as a heater and a temperature detecting unit such as a thermistor are installed at the front end of the insertion portion; and, based on the detection result obtained by the temperature detecting unit, the driving of the heating unit is controlled so as to prevent clouding of the optical members (refer to Japanese Laid-open Patent Publication No. 2014-131531).

### SUMMARY

According to one aspect of the present disclosure, there is provided an endoscope system including: an endoscope including an insertion portion whose front end is inserted inside a subject; a heater disposed at the front end and configured to heat a predetermined member disposed at the front end; a plurality of thermometers disposed near the predetermined member at the front end and configured to detect temperatures at the front end; a power source configured to supply electrical power to the heater; determination circuitry configured to determine whether or not highest temperature, from among the temperatures detected by the plurality of thermometers, is equal to or higher than a first threshold value; and a power controller configured to control, based on determination result of the determination circuitry, the electrical power supplied by the power source to the heater.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram that schematically illustrates an overall configuration of an endoscope system according to an embodiment;

FIG. 2 is a cross-sectional view for explaining an internal configuration of the front end portion of an endoscope illustrated in FIG. 1;

FIG. 3 is a top view of a heating unit illustrated in FIG. 2;

FIG. 4 is a side view of the heating unit illustrated in FIG. 2;

FIG. 5 is a cross sectional view taken along V-V line illustrated in FIG. 4;

FIG. 6 is a block diagram illustrating a functional configuration of the main parts of the endoscope system according to the embodiment;

FIG. 7 is a flowchart for explaining an overview of the operations performed in the endoscope system according to the embodiment;

FIG. 8 is a block diagram illustrating a functional configuration of the main parts of an endoscope system according to a first modification example of the embodiment; and

FIG. 9 is a block diagram illustrating a functional configuration of the main parts of an endoscope system according to a second modification example of the embodiment.

### DETAILED DESCRIPTION

An exemplary embodiment is described below in detail with reference to the accompanying drawings. However, the present disclosure is not limited by the embodiment described below. Moreover, the diagrams referred to in the following explanation illustrate the shapes, the sizes, and the positional relationships only in a schematic manner in order to enable understanding of the details. That is, the present disclosure is not limited by the shapes, the sizes, and the positional relationships illustrated in the drawings. Furthermore, in the explanation with reference to the drawings, identical constituent elements are referred to by the same reference numerals.

Configuration of Endoscope System

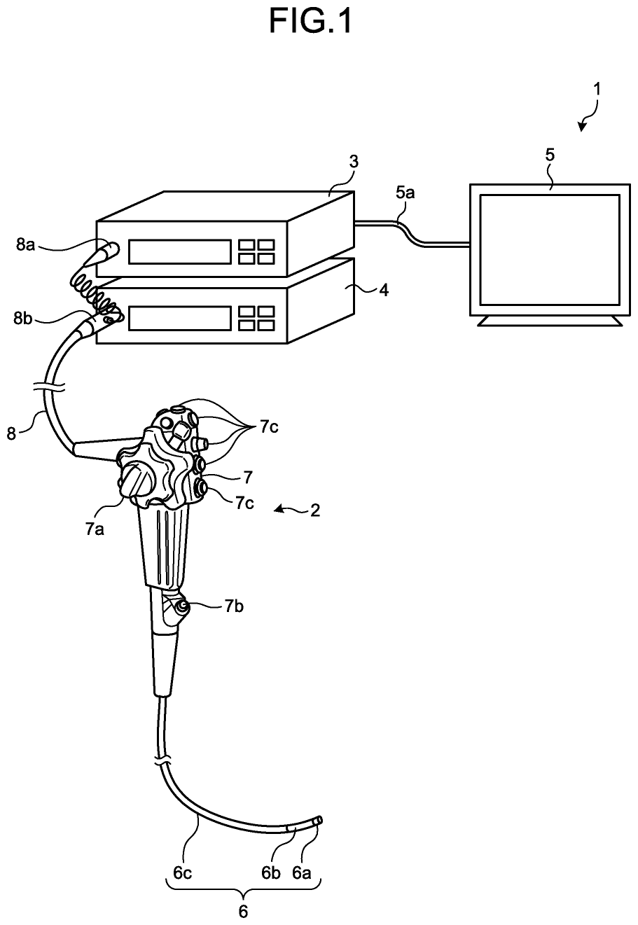

FIG. 1 is a diagram that schematically illustrates an overall configuration of an endoscope system according to the embodiment. An endoscope system 1 illustrated in FIG. 1 includes an endoscope 2 that is inserted inside a subject for taking images of the inside of the body of the subject and generates image signals; a processor 3 that functions as a control unit for performing predetermined image processing with respect to the image signals generated by the endoscope 2 and for controlling the constituent elements of the endoscope system 1; a light source device 4 that generates illumination light to be supplied to the endoscope 2; and a display device 5 that displays images corresponding to the image signals which have been subjected to image processing by the processor 3.

The endoscope 2 includes an insertion portion 6 that is inserted inside a subject; an operating unit 7 that is installed at the proximal end of the insertion portion 6; and a flexible universal cord 8 that extends from the operating unit 7.

The insertion portion 6 is implemented using at least an illumination fiber (a light guiding cable), an electrical cable, and an optical fiber. The insertion portion 6 includes the following: a front end portion 6a that has an imaging device (an imaging unit) (described later) built-in; a freely-bendable curved portion 6b that is made of a plurality of bent pieces; and a flexible tube 6c that is a flexible tube connected to the proximal end of the curved portion 6b. The front end portion 6a has the following components disposed therein: an illuminating unit that irradiates the inside of the subject with the illumination light supplied from the light source device 4 via an illumination lens; an observation portion that generates image signals by taking subject images as a result of light condensation by the optical system; an opening that is communicated with a treatment tool channel; and an insufflation/water supply nozzle.

The operating unit 7 includes the following: a curved knob 7a that is meant for bending the curved portion 6b in the vertical direction and the horizontal direction; a treatment tool insertion portion 7b from which a treatment tool such as a biopsy forceps or a laser knife is insertable inside the body cavity of the subject; and a plurality of switches 7c that enable operations of the peripheral devices such as the light source device 4, an insufflation device, a water supply device, and a gas transportation device. The treatment tool that is inserted from the treatment tool insertion portion 7b passes through an internal treatment tool channel and appears from a forceps opening formed at the front end of the insertion portion 6.

The universal cord 8 is configured using an illumination fiber and an electrical cable. The universal cord 8 is branched at the proximal end thereof, with one of the branched ends representing a connector 8a and the other branched end representing a connector 8b. The connector 8a is detachably attachable to the connector of the processor 3. The connector 8b is detachably attachable to the light source device 4. The universal cord 8 passes on the illumination light, which is supplied from the light source device 4, to the front end portion 6a via the connector 8b and the illumination fiber. Moreover, the universal cord 8 transmits the image signals, which are obtained as a result imaging by the imaging unit (described later), to the processor 3 via the electrical cable and the connector 8a.

The light source device 4 emits light from a light source under the control of the processor 3, and supplies illumination light to the endoscope 2 connected via the connector 8b and the illumination fiber of the universal cord 8. The light source for emitting light is configured using, for example, a light emitting diode (LED) or a xenon lamp and a condenser lens.

The display device 5 displays, via a video cable 5a, a variety of information containing images corresponding to the image signals that have been subjected to predetermined image processing by the processor 3. The display device 5 is configured using a liquid crystal display or an organic electroluminescence (EL) display. Hence, the operator may operate the endoscope 2 while looking at the images (in-vivo images) displayed in the display device 5, and may observe the desired positions inside the subject and determine their characteristics.

Detailed Configuration of Front End Portion of Endoscope

Given below is the explanation of a detailed configuration of the front end portion 6a of the endoscope 2.

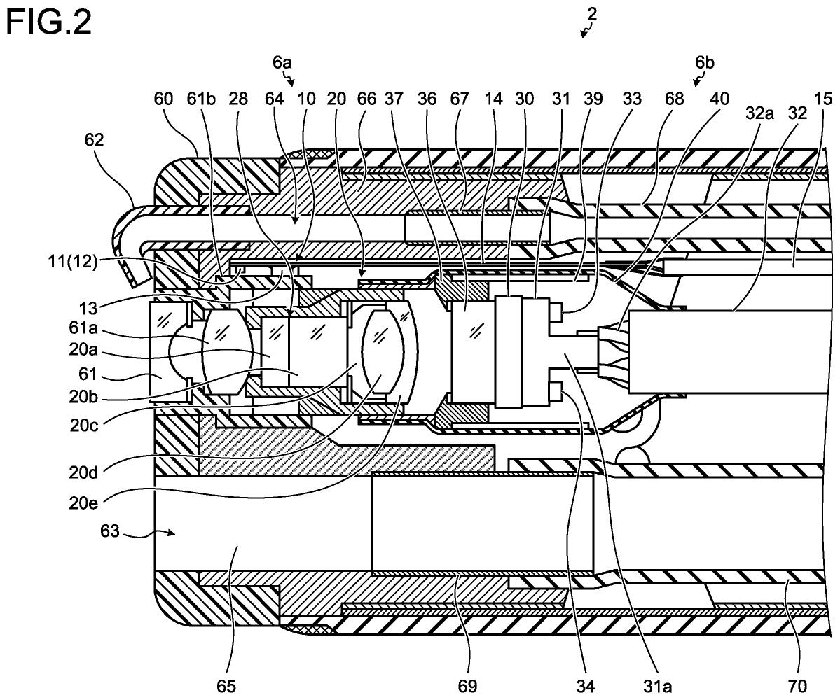

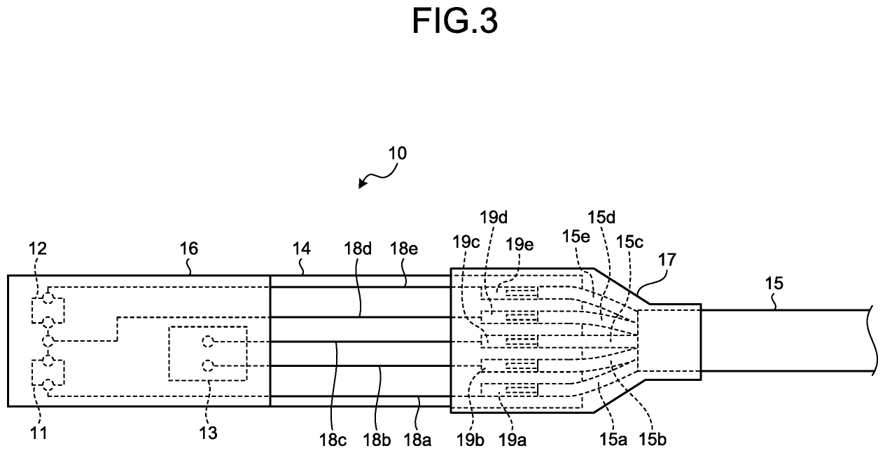

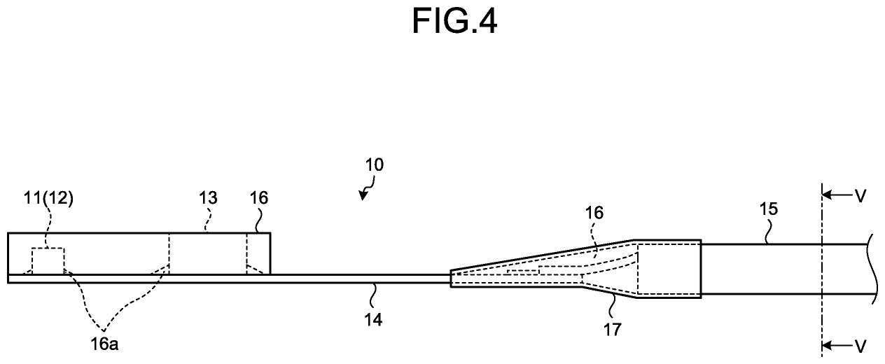

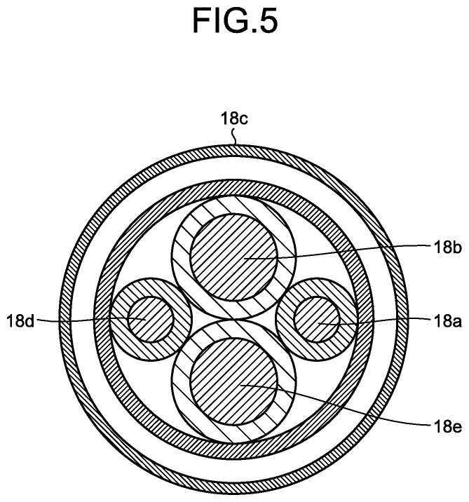

FIG. 2 is a cross-sectional view for explaining an internal configuration of the front end portion 6a of the endoscope 2 illustrated in FIG. 1. FIG. 3 is a top view of a heating unit (described later) illustrated in FIG. 2. FIG. 4 is a side view of the heating unit (described later) illustrated in FIG. 2. FIG. 5 is a cross sectional view taken along V-V line illustrated in FIG. 4.

As illustrated in FIGS. 2 to 5, on the front end portion 6a, a front cover 60 is fit from outside. The front cover 60 is provided with an observation window 61, an illumination lens (not illustrated), an insufflation/water supply nozzle 62, and a forceps opening 63. In a holding portion 61b of the observation window 61, an imaging device 20 that takes images of the inside of the subject via a plurality of lenses including a lens 61a is fit by insertion. On the posterior side of the observation window 61, a front end block 66 is fixedly set in such a way that an insufflation/water supply hole 64 and a forceps insertion hole 65 formed thereon correspond to the nozzle 62 and the forceps opening 63, respectively.

In the rear end portion of the insufflation/water supply hole 64 in the front end block 66, an insufflation/water supply pipe 67 is laid. To the insufflation/water supply pipe 67 is connected an insufflation/water supply tube 68. In the rear end portion of the forceps insertion hole 65, a forceps insertion pipe 69 is disposed. To the forceps insertion pipe 69 is connected a forceps insertion tube 70.

The imaging device 20 (the imaging unit) includes an objective optical unit 28 configured using the following: a plurality of optical lenses 20a to 20e; an image sensor 30 that is disposed on the posterior side of the objective optical unit 28 and that receives the light falling on the objective optical unit 28; a circuit board 31 that is connected to the image sensor 30; and a composite cable 32 that is connected to the image sensor 30 via the circuit board 31 and that transmits the image signals of the subject, which are generated as a result of imaging performed by the image sensor 30, to the processor 3.

On the light receiving surface of the image sensor 30, a cover glass 36 is disposed. On the outer periphery of the cover glass 36, the inner periphery of an image sensor holding frame 37 is fit and is integrally fixed using an adhesive agent.

On the underside of the circuit board 31, an IC 33 and a chip capacitor 34 are installed for converting the image signals, which are received from the image sensor 30, into electrical signals; and a cable 32a of the composite cable 32 is connected to an attaching portion 31a that protrudes on the underside of the circuit board 31.

In the rear end portion of the image sensor holding frame 37, a shield support 39 is disposed to cover the image sensor 30 and the circuit board 31. The outer periphery of the shield support 39 and the image sensor holding frame 37 is covered by a heat-shrinkable tube 40.

In between the holding portion 61b, in which the imaging device 20 is fit by insertion, and the front end block 66, a heating unit 10 is inserted.

The heating unit 10 is disposed in the surrounding area of the imaging device 20 and the observation window 61 functioning as predetermined members, and includes the following: a first temperature detecting unit 11 that detects temperature information of the front end portion 6a; a second temperature detecting unit 12 that is disposed parallel to the first temperature detecting unit 11 along the circumferential direction around the optical axis of the objective optical unit 28 and that detects temperature information of the front end portion 6a; and a heating portion 13 that heats predetermined members such as the observation window 61 and the lens 61a. In the present embodiment, it is also possible to dispose a plurality of first temperature detecting units 11 and a plurality of second temperature detecting units 12 in the circumferential direction around the optical axis of the objective optical unit 28. That is, according to the embodiment, as a result of arranging a plurality of temperature sensors in a circular shape, the diameter of the front end portion 6a may be prevented from becoming larger. The first temperature detecting unit 11 and the second temperature detecting unit 12 are configured using, for example, negative temperature coefficient (NTC) thermistors. Meanwhile, in the present embodiment, the first temperature detecting unit 11 need not be limited to be NTC thermistors, and alternatively positive temperature coefficient (PTC) thermistors may be used. Moreover, the first temperature detecting unit 11 and the second temperature detecting unit 12 may be configured to have mutually different characteristic features.

An FPC board 14 has the length spanning from the front end portion 6a to the curved portion 6b, and is disposed in such a way that the front end thereof is positioned in the vicinity of optical members such as the observation window 61, the lens 61a, and the optical lenses 20a to 20e. The first temperature detecting unit 11, the second temperature detecting unit 12, and the heating portion 13 are installed in the vicinity of the front end side of the flexible printed circuit board 14 (hereinafter, referred to as the “FPC board 14”), that is, in the vicinity of the optical members; and the surrounding portion of their connections are protected by an underfill material 16a. Moreover, the FPC board 14 on which the first temperature detecting unit 11, the second temperature detecting unit 12, and the heating portion 13 are installed is sealed on top by an encapsulation resin 16. At the proximal end of the FPC board 14 that extends to the curved portion 6b; connecting electrodes 19a to 19e are formed with cables 15a to 15e, respectively, of a composite cable 15 connected thereto. The outer periphery of the FPC board 14 at which the cables 15a to 15e are connected is covered by a heat-shrinkable tube 17, and the internal portion thereof is sealed by the encapsulation resin 16.

The first temperature detecting unit 11 and the second temperature detecting unit 12 are parallel circuits connected to the cables 15a, 15d, and 15e via wirings 18a, 18d, and 18e, respectively, and via the connecting electrodes 19a, 19d, and 19e, respectively. The heating portion 13 is an independent heater circuit connected to the cables 15b and 15c via wirings 18b and 18c, respectively, and via connecting electrodes 19b and 19c, respectively.

In the heating unit 10 configured in the manner explained above, the heating portion 13, whose top surface is exposed from the encapsulation resin 16, abuts against the holding portion 61b and thus gets fixed. In the FPC board 14, the end portion at the proximal end (i.e., the side to which the composite cable 15 is connected) is adjusted to have such a length that it gets positioned in the vicinity of the border between the front end portion 6a and the curved portion 6b.

Functional Configuration of Main Parts of Endoscope System Including Heating Unit at Front End Portion

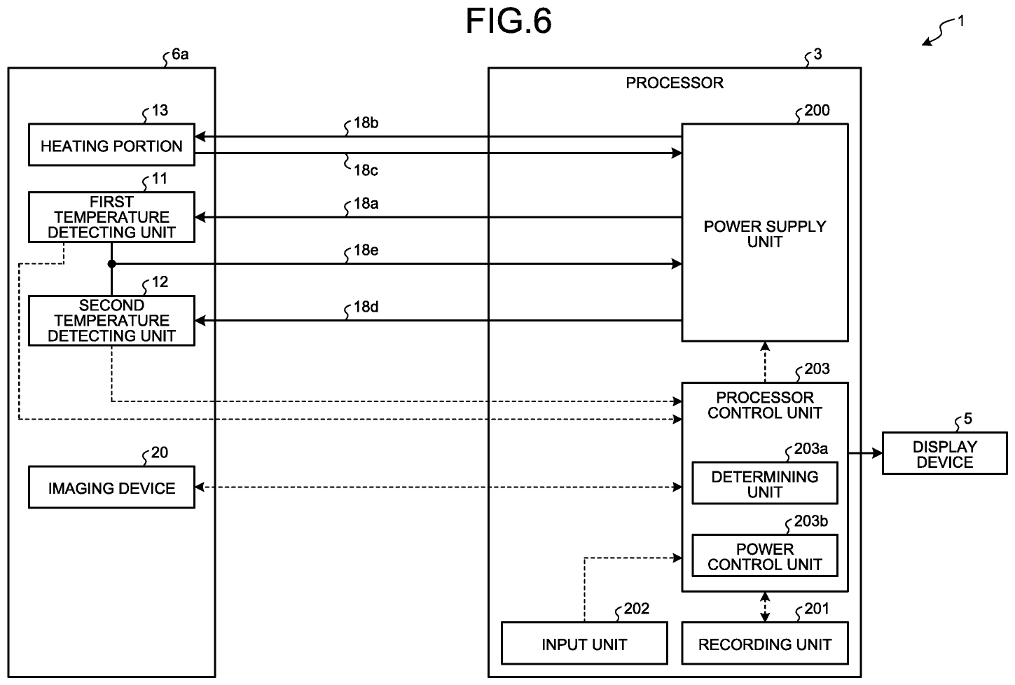

Given below is the explanation of a functional configuration of the main parts of the endoscope system 1 including the heating unit 10 at the front end portion 6a. FIG. 6 is a block diagram illustrating a functional configuration of the main parts of the endoscope system 1. Meanwhile, since the configuration of the front end portion 6a is already explained with reference to FIGS. 3 to 5, the detailed explanation thereof is not again given with reference to FIG. 6; and the following explanation is given about a functional configuration of the main parts of the processor 3.

As illustrated in FIG. 6, the processor 3 includes a power supply unit 200, a recording unit 201, an input unit 202, and a processor control unit 203.

The power supply unit 200 supplies electrical power to the first temperature detecting unit 11, the second temperature detecting unit 12, and the heating portion 13 under the control of the processor control unit 203. The power supply unit 200 is configured using a regulator that performs voltage adjustment with respect to the voltage input from outside.

The recording unit 201 is used to record various programs to be executed by the endoscope system 1, and to record the data being processed. The recording unit 201 is configured using a volatile memory or a nonvolatile memory.

The input unit 202 is configured using input interfaces such as a keyboard, switches, buttons, and a touch-sensitive panel. The input unit 202 receives input of instruction signals according to operations performed from outside, and outputs the instruction signals to the processor control unit 203.

The processor control unit 203 comprehensively controls the components of the endoscope system 1. The processor control unit 203 is configured using a central processing unit (CPU). The processor control unit 203 includes a determining unit 203a and a power control unit 203b.

When the power supply unit 200 is supplying electrical power to the heating portion 13 (i.e., when the heating portion 13 is in the heating state), the determining unit 203a determines whether or not the highest temperature from among a plurality of temperature values detected by the first temperature detecting unit 11 and the second temperature detecting unit 12 is equal to or higher than a first threshold value T_{P}. Moreover, when the power supply unit 200 has stopped supplying electrical power to the heating portion 13 (i.e., when the heating portion 13 is in a halt state), the determining unit 203a determines whether or not the lowest temperature from among the temperature values detected by the first temperature detecting unit 11 and the second temperature detecting unit 12 is lower than a second threshold value T_{Q} that is smaller than the first threshold value T_{P} (i.e., T_{P}>T_{Q} holds true).

Based on the determination result obtained by the determining unit 203a, the power control unit 203b controls the electrical power supplied by the power supply unit 200 to the heating portion 13. More particularly, when the power supply unit 200 is supplying electrical power to the heating portion 13, if the determining unit 203a determines that the highest temperature is equal to or higher than the first threshold value T_{P}, the power control unit 203b makes the power supply unit 200 stop supplying electrical power to the heating portion 13. On the other hand, if the determining unit 203a determines that the highest temperature is not equal to or higher than the first threshold value T_{P}, the power control unit 203b makes the power supply unit 200 continue supplying electrical power to the heating portion 13. Moreover, when the power supply unit 200 has stopped supplying electrical power to the heating portion 13, if the determining unit 203a determines that the lowest temperature is lower than the second threshold value T_{Q}, the power control unit 203b makes the power supply unit 200 start supplying electrical power to the heating portion 13. On the other hand, if the determining unit 203a determines that the lowest temperature is not lower than the second threshold value T_{Q}, the power control unit 203b makes the power supply unit 200 continue with the stoppage in the supply of electrical power to the heating portion 13.

Operations in Endoscope System

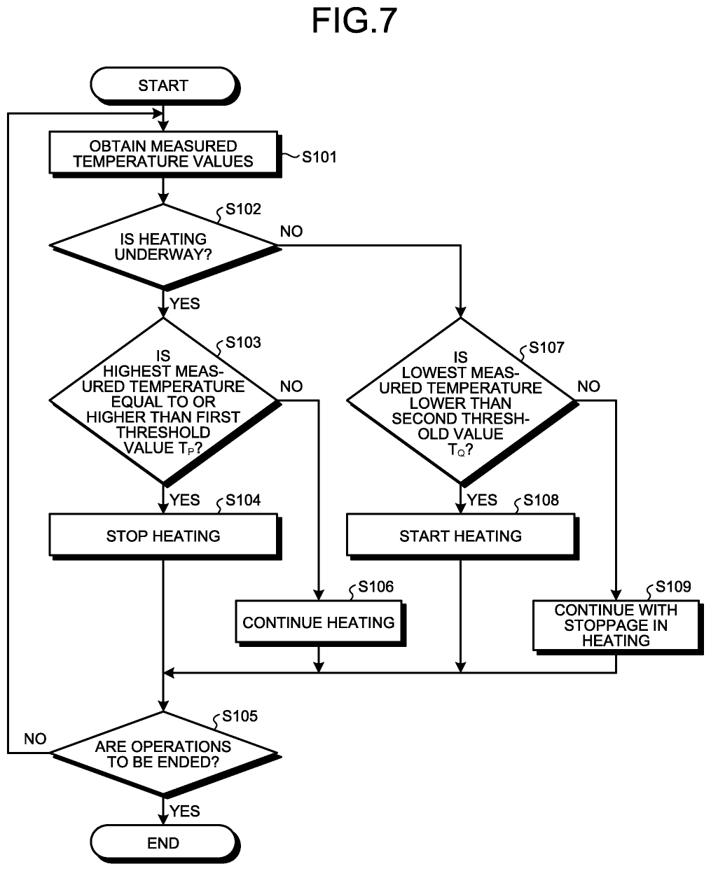

Given below is the explanation of the operations performed in the endoscope system 1. FIG. 7 is a flowchart for explaining an overview of the operations performed in the endoscope system 1. With reference to FIG. 7, from among the operations performed in the endoscope system 1, the explanation is given only about the temperature control performed with respect to the heating portion 13.

As illustrated in FIG. 7, firstly, the determining unit 203a obtains the measured temperature values detected by the first temperature detecting unit 11 as well as the second temperature detecting unit 12 (Step S101).

Then, the determining unit 203a determines whether or not the heating portion 13 is in the heating state (Step S102). More particularly, the determining unit 203a determines whether or not the power supply unit 200 is supplying electrical power to the heating portion 13. If the determining unit 203a determines that the heating portion 13 is in the heating state (Yes at Step S102), then the system control proceeds to Step S103 (described later). On the other hand, if the determining unit 203a determines that the heating portion 13 is not in the heating state (No at Step S102), then the system control proceeds to Step S107 (described later).

At Step S103, the determining unit 203a determines whether or not the highest measured temperature from among the measured temperature values obtained by the first temperature detecting unit 11 and the second temperature detecting unit 12 is equal to or higher than the first threshold value T_{P}. If the determining unit 203a determines that the highest measured temperature from among the measured temperature values obtained by the first temperature detecting unit 11 and the second temperature detecting unit 12 is equal to or higher than the first threshold value T_{P} (Yes at Step S103), then the system control proceeds to Step S104 (described later). On the other hand, if the determining unit 203a determines that the highest measured temperature from among the measured temperature values obtained by the first temperature detecting unit 11 and the second temperature detecting unit 12 is not equal to or higher than the first threshold value T_{P} (No at Step S103), then the system control proceeds to Step S106 (described later).

At Step S104, the power control unit 203b makes the power supply unit 200 stop supplying electrical power to the heating portion 13, and thus stops the heating attributed to the heating portion 13.

Then, if an instruction signal for ending the examination of the subject is input via the input unit 202 (Yes at Step S105), then the endoscope system 1 ends the operations. On the other hand, if an instruction signal for ending the examination of the subject is not input via the input unit 202 (Yes at Step S105), then the system control returns to Step S101.

At Step S106, the power control unit 203b makes the power supply unit 200 continue supplying electrical supply to the heating portion 13, and thus continues with the heating attributed to the heating portion 13. After Step S106, the system control returns to Step S105.

At Step S107, the determining unit 203a determines whether or not the lowest measured temperature from among the measured temperature values obtained from the first temperature detecting unit 11 and the second temperature detecting unit 12 is lower than the second threshold value T_{Q}. If the determining unit 203a determines that the lowest measured temperature from among the measured temperature values obtained from the first temperature detecting unit 11 and the second temperature detecting unit 12 is lower than the second threshold value T_{Q} (Yes at Step S107), then the system control proceeds to Step S108. On the other hand, if the determining unit 203a determines that the lower measured temperature from among the measured temperature value obtained from the first temperature detecting unit 11 and the second temperature detecting unit 12 is not lower than the second threshold value T_{Q} (No at Step S107), then the system control proceeds to Step S109.

At Step S108, the power control unit 203b makes the power supply unit 200 supply electrical power to the heating portion 13 and thus starts the heating attributed to the heating portion 13 that was in the halt state. After Step S108, the system control returns to Step S105.

At Step S109, the power control unit 203b makes the power supply unit 200 continue with the stoppage in the supply of electrical power to the heating portion 13, and thus keeps the heating portion 13 in the halt state. After Step S109, the system control returns to Step S105.

According to the embodiment as described above, based on the detection result obtained by the determining unit 203a, the power control unit 203b controls the electrical power supplied by the power supply unit 200 to the heating portion 13. Hence, even when there is a change in the temperature characteristics of the first temperature detecting unit 11 and the second temperature detecting unit 12, the heating portion 13 may be controlled with accuracy.

Moreover, according to the embodiment, when the power supply unit 200 is supplying electrical power to the heating portion 13, if the determining unit 203a determines that the highest temperature is equal to or higher than the first threshold value T_{P}, the power control unit 203b stops the supply of electrical power from the power supply unit 200 to the heating portion 13. On the other hand, if the determining unit 203a determines that the highest temperature is not equal to or higher than the first threshold value T_{P}, the power control unit 203b continues with the supply of electrical power from the power supply unit 200 to the heating portion 13. Hence, even if there is a change in the temperature characteristics of the first temperature detecting unit 11 and the second temperature detecting unit 12, the front end portion 6a may be prevented from getting excessively heated. That is, since the power control unit 203b does not perform control on the basis of the lowest temperature, the front end portion 6a may be prevented from getting excessively heated.

Furthermore, according to the embodiment, when the power supply unit 200 has stopped supplying electrical power to the heating portion 13, if the determining unit 203a determines that the lowest temperature is lower than the second threshold value T_{Q}, the power control unit 203b makes the power supply unit 200 start supplying electrical power to the heating portion 13. On the other hand, if the determining unit 203a determines that the lowest temperature is not lower than the second threshold value T_{Q}, the power control unit 203b makes the power supply unit 200 continue with the stoppage in the supply of electrical power to the heating portion 13. Hence, even when there is a change in the temperature characteristics of the first temperature detecting unit 11 and the second temperature detecting unit 12, it becomes possible to prevent an excessive drop in temperature of the front end portion 6a. That is, since the power control unit 203b does not perform control on the basis of the highest temperature, it becomes possible to prevent an excessive drop in temperature of the front end portion 6a.

Moreover, according to the embodiment, based on the determination result obtained by the determining unit 203a, the power control unit 203b controls the electrical power supplied by the power supply unit 200 to the heating portion 13. Hence, even when there are individual differences between the first temperature detecting unit 11 and the second temperature detecting unit 12 or when there is some malfunctioning in the first temperature detecting unit 11 and the second temperature detecting unit 12, the heating portion 13 may be controlled with accuracy.

Furthermore, according to the embodiment, although the first temperature detecting unit 11 and the second temperature detecting unit 12 are installed in the front end portion 6a, that is not the only possible case. Alternatively, it is possible to install a plurality of temperature detecting units. In that case, the temperature detecting units may be installed in the circumferential direction around the optical axis of the objective optical unit 28.

Moreover, in the embodiment, although the first temperature detecting unit 11 and the second temperature detecting unit 12 have the same characteristic features, that is not the only possible case and alternatively they may be configured to have different characteristic features. For example, in the case of configuring the first temperature detecting unit 11 and the second temperature detecting unit 12 using thermistors, it is possible to use thermistors having mutually different breakdown behaviors. More particularly, in the case of using NTC thermistors, they may have different layer counts of the internal layer structure and may have different layer structures. Of course, in the case of using NTC thermistors, mutually different materials may be used.

### First Modification Example

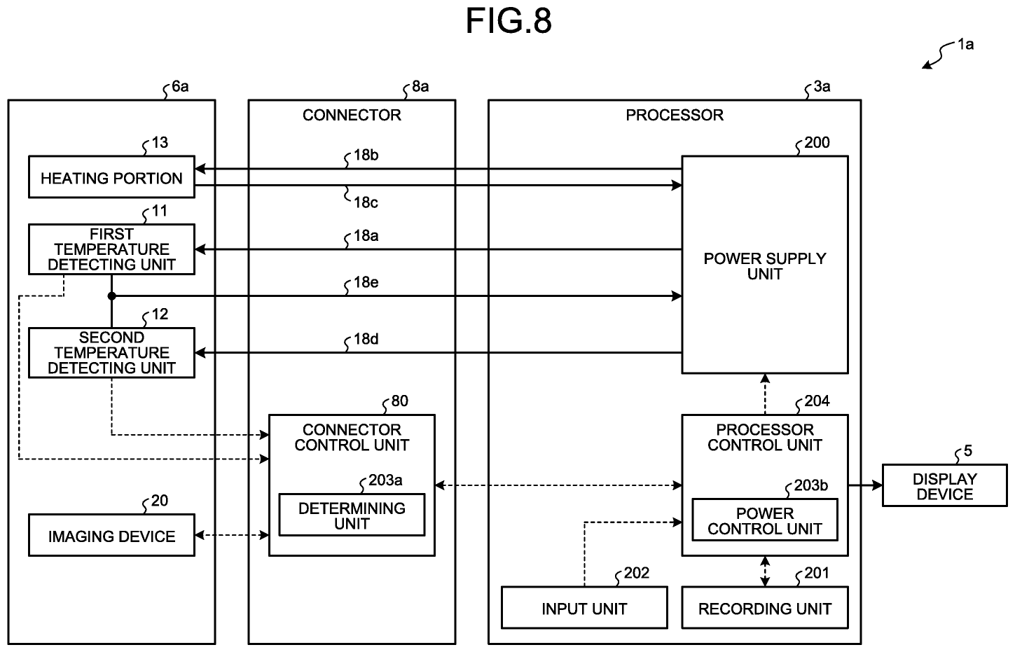

Given below is the explanation of a first modification example of the embodiment. FIG. 8 is a block diagram illustrating a functional configuration of the main parts of an endoscope system according to the first modification example of the embodiment. In the following explanation, the identical constituent elements to the endoscope system 1 according to the embodiment are referred to by the same reference numerals, and their explanation is not repeated.

An endoscope system la illustrated in FIG. 8 includes a processor 3a in place of the processor 3 according to the embodiment. Moreover, the connector 8a includes a connector control unit 80.

The connector control unit 80 includes the determining unit 203a according to the embodiment. The connector control unit 80 is configured using a field-programmable gate array (FPGA).

The processor 3a includes a processor control unit 204 in place of the processor control unit 203 of the processor 3 according to the embodiment. The processor control unit 204 includes the power control unit 203b.

According to the first modification example of the embodiment, it becomes possible to achieve the same effects as achieved in the embodiment; and, even when there is a change in the temperature characteristics of the first temperature detecting unit 11 and the second temperature detecting unit 12, the heating portion 13 may be controlled with accuracy.

Meanwhile, in the first modification example of the embodiment, although the determining unit 203a is disposed in the connector 8a, that is not the only possible case. Alternatively, the determining unit 203a may be disposed inside the operating unit 7.

### Second Modification Example

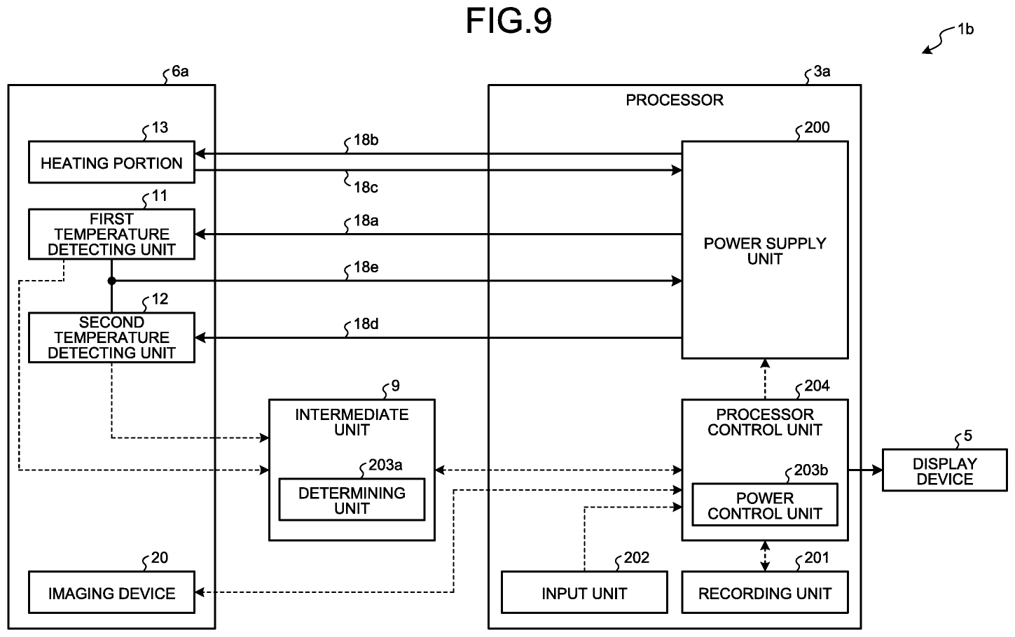

Given below is the explanation of a second modification example of the embodiment. FIG. 9 is a block diagram illustrating a functional configuration of the main parts of an endoscope system according to the second modification example of the embodiment. In the following explanation, the identical constituent elements to the endoscope system 1 according to the embodiment are referred to by the same reference numerals, and their explanation is not repeated.

An endoscope system 1b illustrated in FIG. 9 includes the processor 3a and an intermediate unit 9 in place of the processor 3 according to the embodiment. Moreover, the intermediate unit 9 includes the determining unit 203a.

According to the second modification example of the embodiment, it becomes possible to achieve the same effects as achieved in the embodiment; and, even when there is a change in the temperature characteristics of the first temperature detecting unit 11 and the second temperature detecting unit 12, the heating portion 13 may be controlled with accuracy.

### Other Embodiments

A plurality of constituent elements disclosed in the embodiment may be appropriately combined and various inventions may be made. For example, some of the constituent elements mentioned in the embodiment may be deleted. Moreover, the constituent elements mentioned in the embodiment may be appropriately combined.

In the embodiment, although the processor and the light source device are configured to be different components, they may alternatively be configured in an integrated manner.

Moreover, the term “unit” mentioned above may be read as “device” or “circuit”. For example, a control unit may be read as a control device or a control circuit.

In the embodiment, although the endoscope system includes a flexible endoscope, the present invention may be implemented also in an endoscope system including a rigid endoscope or an endoscope system including an industrial endoscope.

Meanwhile, in the explanation of the flowchart given in the present written description, the context is explicitly illustrated using expressions such as “firstly”, “then”, and “subsequently”. However, the sequence of operations required to implement the present invention are not uniquely fixed by those expressions. That is, the sequence of operations performed in the flowchart given in the present written description may be varied without causing contradiction.

According to the present invention, even when there is a change in the temperature characteristics of the temperature detecting units, the heating portion may be controlled with accuracy.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
an endoscope including an insertion portion whose front end is inserted inside a subject;
a heater disposed at the front end and configured to heat a predetermined member disposed at the front end;
a plurality of thermometers disposed near the predetermined member at the front end and configured to detect temperatures at the front end;
a power source configured to supply electrical power to the heater;
determination circuitry configured to determine whether or not highest temperature, from among the temperatures detected by the plurality of thermometers, is equal to or higher than a first threshold value; and
a power controller configured to control, based on determination result of the determination circuitry, the electrical power supplied by the power source to the heater.

2. The endoscope system according to claim 1, wherein the power controller is configured to control the power source to stop supplying the electrical power to the heater when the determination circuitry determines that the highest temperature is equal to or higher than the first threshold value in a state that the power source is supplying the electrical power to the heater.

3. The endoscope system according to claim 2, wherein the power controller is configured to control the power source to continue supplying the electrical power to the heater when the determination circuitry determines that the highest temperature is lower than the first threshold value in the state that the power source is supplying the electrical power to the heater.

4. The endoscope system according to claim 2, wherein
the determination circuitry is configured to determine whether or not lowest temperature from among the temperatures is lower than a second threshold value that is smaller than the first threshold value when the power source has stopped supplying the electrical power to the heater, and
the power controller is configured to control the power source to start supplying the electrical power when the determination circuitry determines that the lowest temperature is lower than the second threshold value.

5. The endoscope system according to claim 4, wherein the power controller is configured to continue with stoppage in supply of the electrical power when the determination circuitry determines that the lowest temperature is not lower than the second threshold value.

6. An endoscope system according to claim 2, further comprising a processor to which the endoscope is connected in a detachable manner, the processor is configured to perform image processing with respect to image signal generated by the endoscope, wherein
the power source, the determination circuitry and the power controller are disposed in the processor.

7. The endoscope system according to claim 2, further comprising a processor configured to perform image processing with respect to image signal generated by the endoscope, wherein
the endoscope further includes a connector configured to enable connection with the processor in a detachable manner,
the power source and the power controller are disposed in the processor, and
the determination circuitry is disposed in the connector.

8. A method of controlling an endoscope system, the method comprising:
detecting temperature at an front end of the endoscope by a plurality of thermometers disposed at the front end;
determining whether or not highest temperature, from among a plurality of temperatures detected by the plurality of thermometers, is equal to or higher than a first threshold value; and
controlling, based on determination result, electrical power supplied to a heater disposed at the front end and configured to heat a predetermined member disposed at the front end.

9. A processor for being connected to an endoscope including a heater configured to heat a predetermined member disposed at a front end of the endoscope, and a plurality of thermometers configured to detect temperatures at the front end, the processor comprising:
a power source configured to supply electrical power to the heater;
determination circuitry configured to determine whether or not highest temperature, from among the temperatures detected by the plurality of thermometers, is equal to or higher than a first threshold value; and
a power controller configured to control, based on determination result of the determination circuitry, the electrical power supplied by the power source to the heater.

10. An endoscope comprising:
an insertion portion whose front end is inserted inside a subject;
a plurality of thermometers disposed near a predetermined member disposed at the front end and configured to detect temperatures at the front end, the temperatures detected by the plurality of thermometers being output to determination circuitry configured to determine whether or not highest temperature, from among the temperatures, is equal to or higher than a first threshold value; and
a heater disposed at the front end and configured to heat the predetermined member by electrical power, the electrical power being controlled and supplied based on determination result of the determination circuitry.
